(19) United States
(12) Patent Application Publication
(10) Pub. No.: US 2004/0228911 A1
(43) Pub. Date: Nov. 18, 2004

(54) Vinorelbine compositions and methods of use

(75) Inventors: Jia-Ai Zhang, Vernon Hills, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL

(21) Appl. No.: 10/786,866

(22) Filed: Feb. 24, 2004

Related U.S. Application Data
(63) Continuation of application No. PCT/US02/26907, filed on Aug. 23, 2002.
(60) Provisional application No. 60/314,959, filed on Aug. 24, 2001.

(51) Int. Cl. A61K 31/4745; A61K 9/127; A61K 9/48

(52) U.S. Cl. 424/450; 514/283

(57) ABSTRACT

The present invention is for novel compositions and methods for treating cancer, particularly, for treating cancer in mammals and more particularly in humans. The therapeutic compositions of the present invention include liposome entrapped vinorelbine in which the liposome can contain any of a variety of neutral or charged liposome-forming compounds and cardiolipin. The liposomes of the present invention can be either multilamellar vesicles or unilamellar vesicles, as desired.

### CROSS-REFERENCE TO RELATED APPLICATIONS

This patent application is a continuation of co-pending PCT Application No. PCT/US02/26907, filed on Aug. 23, 2002, which claims the benefit of U.S. Provisional Patent Application No. 60/314,959, filed on Aug. 24, 2001. All prior applications are hereby incorporated by reference in their entireties.

### FIELD OF THE INVENTION

This invention pertains to formulations and methods for making and using vinorelbine-containing liposomes.

### DESCRIPTION OF THE BACKGROUND

Vinca alkaloids are well known to be useful as therapeutic agents in the treatment of cancer. They are comprised of the two multiringed moieties, vindoline and catharanthine. Vinorelbine is a semi-synthetic vinca alkaloid derivative in, unlike other vinca alkaloids, the catharanthine moiety is the site of modification. The chemical name for vinorelbine is 3′,4′-didehydro-4′-deoxy-C-norvincaleukoblastine [R—(R,R)-2,3-dihydroxybutanedioate (1:2)(salt)]. Vinorelbine tartrate is a white to yellow or light brown amorphous powder with the molecular formula C₄₅H₅₄N₄O₈-2C₄H₆O₆ and molecular weight of 1079.12. The aqueous solubility is less than 1000 mg/mL in distilled water.

The U.S. Food and Drug Administration (FDA) first approved vinorelbine hydrochloride for sale in the United States in 1994 as an injectable formulation under the tradename NAVELBINE®. NAVELBINE® is indicated for use as a single agent or in combination with cisplatin for the first-line treatment of ambulatory patients with unresectable, advanced non-small cell lung cancer (NSCLC). In patients with Stage IV non small cell lung cancer (NSCLC), NAVELBINE® is indicated as a single agent or in combination with cisplatin. In Stage III NSCLC, NAVELBINE® is indicated in combination with cisplatin. NAVELBINE® is also available for the treatment of metastatic breast cancer. NAVELBINES® (vinorelbine tartrate) injection is for intravenous administration. Each vial contains vinorelbine tartrate equivalent to 10 mg (1 mL vial) or 50 mg (5 mL vial) vinorelbine in Water for Injection. No preservatives or other additives are present. The aqueous solution is sterile and nonpyrogenic. The pH of NAVELBINE® injection is approximately 3.5.

Vinorelbine is thought to block the division of growing cells by binding to tubulin and interfering with microtubule assembly, thereby preventing or interfering with mitosis at metaphase. As with other vinca alkaloids, vinorelbine possibly also interferes with: 1) amino acid, cyclic AMP, and glutathione metabolism, 2) calmodulin-dependent Ca⁺⁺ transport ATPase activity, 3) cellular respiration, and 4) nucleic acid and lipid biosynthesis. In intact tectal plates from mouse embryos, vinorelbine, vincristine, and vinblastine inhibited mitotic microtubule formation at the same concentration (2 μM), inducing a blockade of cells at metaphase. Vincristine produced depolymerization of axonal microtubules at 5 μM, but vinblastine and vinorelbine did not have this effect until concentrations of 30 μM and 40 μM, respectively. These data suggest relatively selective binding of vinorelbine to mitotic microtubules.

Unfortunately, the toxicity of vinorelbine limits the dosage of drug that can be administered to patients. Moreover, the development of multidrug resistance in cells exposed to vinorelbine further limits its effectiveness. Consequently, formulations of vinorelbine are needed that limit the toxicity of vinorelbine and that minimize multidrug resistance in treated cells.

### SUMMARY OF THE INVENTION

The present invention provides for novel vinorelbine compositions, their preparation methods, and their use in treating proliferative diseases such as cancer, particularly in mammals, especially in humans. The compositions of the present invention include liposome-entrapped vinorelbine in which the liposome can contain any of a variety of neutral or charged liposome-forming materials and cardiolipin. The liposome forming material is an amphiphilic molecule such as phosphatidylcholine, cholesterol, dipalmitoylphosphatidylcholine, phosphatidylserine, and the like. The cardiolipin in the liposomes can be derived from natural sources or synthetic. Depending on their composition, the liposomes can carry net negative or positive charges or can be neutral. Preferred liposomes also contain α-tocopherol.

The liposomal compositions can be used advantageously in conjunction with secondary therapeutic agents other than vinorelbine, including antineoplastic, antifungal, antibiotic among other active agents, particularly cisplatin. The liposomes can be multilamellar vesicles, unilamellar vesicles, or their mixtures as desired. The invention specifically contemplates methods in which a therapeutically effective amount of the inventive liposomes in a pharmaceutically acceptable excipient are administered to a mammal, such as a human.

Desirably, the composition and method present one or more of the following advantages: 1) avoidance of solubility problems, 2) high vinorelbine and liposome stability, 3) ability to administer vinorelbine as a bolus or short infusion in a high concentration, 4) reduced vinorelbine toxicity 5) increased therapeutic efficacy of vinorelbine, and 6) modulation of multidrug resistance in cancer cells. These and other properties and advantages of the present invention will be apparent upon reading the following detailed description.

### DETAILED DESCRIPTION OF THE PREFERRED EMBODIMENTS

Generally, the liposomes for use in the present invention can be formed by known techniques. For example, in one preferred technique vinorelbine is dissolved in a hydrophobic solvent with cardiolipin and the cardiolipin allowed to form complexes with vinorelbine. The cardiolipin/vinorelbine-containing mixture can be evaporated to form a film in order to facilitate complex formation. Thereafter, solutions containing any desired additional lipophilic ingredients can be added to the film and the vinorelbine/cardiolipin complexes dissolved or thoroughly dispersed in the solution. The solution can then be evaporated to form a second lipid film. A polar solvent, such as an aqueous solvent, can then be added to the lipid film and the resulting mixture vigorously homogenized to produce the present inventive liposomes. In another preferred technique, all of the lipophilic ingredients can be dissolved in a suitable solvent that can then be evaporated to form a lipophilic film. A polar solvent, such as an aqueous solvent, can then be added to the lipid film and the resulting mixture vigorously homogenized to produce the present inventive liposomes. In yet another alternative method, vinorelbine can be dissolved in a suitable aqueous solvent or buffers. The aqueous of vinorelbine can then be added to the lipid film and the resulting mixture vigorously homogenized to produce liposomes, emulsions and micelles, as desired.

Where the vinorelbine is dissolved in the lipid film as described above, the dosage form can be conveniently packaged in a single vial to which a suitable aqueous solution can be added to form the liposomes. Alternatively, a two vial system can be prepared in which the lipophilic ingredients are contained as a film in one vial and aqueous ingredients containing vinorelbine are provided in a second vial. The aqueous vinorelbine-containing ingredients can be transferred to the vial containing the lipid film, and the liposomes formed by standard methods.

In a preferred embodiment, the liposomes, once formed, can be filtered through suitable filters to control their size distribution. Suitable filters include those that can be used to obtain the desired size range of liposomes from a filtrate. For example, the liposomes can be formed and, thereafter, filtered through a 5 micron filter to obtain liposomes having a diameter of about 5 microns or less. Alternatively, 1 μm, 500 nm, 100 nm, or other suitable filters can be used to obtain liposomes of desired size.

In accordance with the invention vinorelbine is dissolved in a suitable solvent. Suitable solvents are those in which vinorelbine is soluble and which can be evaporated without leaving a pharmaceutically unacceptable residue. For example, non-polar or slightly polar solvents may be used, such as ethanol, methanol, chloroform, methylene chloride, or acetone.

Any suitable cardiolipin preparation can be used in the present invention. For example, cardiolipin can be purified from natural sources or can be chemically synthesized, such as tetramyristylcardiolipin, by such methods as are known in the art. Cardiolipin can be dissolved in a suitable solvent as described above for vinorelbine and the solutions mixed or the cardiolipin can be dissolved directly with vinorelbine.

In addition to cardiolipin, any suitable liposome-forming material can be used in the present liposomes. Suitable liposome forming materials include synthetic, semi-synthetic (modified natural) or naturally occurring compounds having a hydrophilic portion and a hydrophobic portion. Such compounds are amphiphilic molecules and can have net positive, negative, or neutral charges. The hydrophobic portion of liposome forming compounds can include one or more nonpolar, aliphatic chains, for example, palmitoyl groups. Examples of suitable liposome-forming compounds include phospholipids, sterols, fatty acids, and the like. Preferred liposome forming compounds include cardiolipin, phosphatidylcholine, cholesterol, dipalmitoyl phosphatidylcholine, phosphatidyl serine, and α-tocopherol.

As described above for cardiolipin and vinorelbine, the liposome-forming material can be dissolved in a suitable solvent, which can be a low polarity solvent such as chloroform, or a non-polar solvent, such as n-hexane. Other lipophilic ingredients can be admixed with the aforementioned ingredients, the ingredients can then be mixed with vinorelbine and the solvent evaporated to produce a homogeneous lipid film. Solvent evaporation can be by any suitable means that preserves the stability of vinorelbine and other lipophilic ingredients.

Liposomes can then be formed by adding a polar solution, preferably an aqueous solution, such as a saline solution, to the lipid film and dispersing the film by vigorous mixing. Optionally, the polar solution can contain vinorelbine. The solution can be pure water, or it can contain salts, buffers, or other soluble active agents. Any method of mixing can be used provided that the chosen method induces sufficient shearing forces between the lipid film and polar solvent to strongly homogenize the mixture and form liposomes. For example, mixing can be by vortexing, magnetic stirring, and/or sonicating. Multilamellar liposomes can be formed simply by vortexing the solution. Where unilamellar liposomes are desired a sonication or filtration step is included in the process.

More generally, any suitable method of forming liposomes can be used so long as it provides liposome entrapped vinorelbine. Thus, solvent evaporation methods that do not involve formation of a dry lipid film can be used. For example, liposomes can be prepared by forming an emulsion in an aqueous and organic phase and evaporating the organic solvent. Reverse-phase evaporation, infusion procedures, and detergent dilution, can be used to produce the liposomes. The present invention is intended to encompass liposome-entrapped vinorelbine, without regard to the procedure for making the liposomes.

Suitable liposomes can be neutral, negatively, or positively charged, the charge being a function of the charge of the liposome components and pH of the liposome solution. For example, at neutral pH, positively charged liposomes can be formed from a mixture of phosphatidylcholine, cholesterol, and stearyl amine. Alternatively, negatively charged liposomes can be formed from phosphatidylcholine, cholesterol, and phosphatidyl serine.

The preferred liposome entrapped vinorelbine compositions contains suitable amounts of vinorelbine. Suitable amounts can include from 1 to 50 wt. % vinorelbine, and more preferably 2 to 25 wt. % vinorelbine. Preferred compositions also contain cardiolipin, cholesterol, phosphatidylcholine, and α-tocopherol in suitable amounts. The inventive compositions can contain any suitable amount of cardiolipin. Suitable amounts can include from 1 to 50 wt. % cardiolipin, and more preferably 2 to 25 wt. % cardiolipin. The inventive compositions can contain any suitable amount of phosphatidylcholine. Suitable amounts of phosphatidylcholine can include from 1 to 95 wt. % cardiolipin, and more preferably 20 to 75 wt. % phosphatidylcholine. Preferred liposomes of the present invention also contain suitable amounts of α-tocopherol or other suitable antioxidants. Suitable amounts range from 0.001 wt. % to 10 wt. % α-tocopherol, such as, for example, 5 wt. % α-tocopherol. For reference, wt. % refers to the relative mass of each ingredient in the final composition without regard to the amount of added water.

To improve shelf-life and preserve liposome stability, the present invention provides vinorelbine liposome preparations which can be stored for extended periods of time without substantial leakage from the liposomes of internally encapsulated materials.

The present invention provides a vinorelbine liposome preparations which can be dehydrated, stored for extended periods of time while dehydrated, and then rehydrated when and where they are to be used, without losing a substantial portion of loaded vinorelbine during the dehydration, storage, and rehydration processes. To achieve these and other objects, the invention, in accordance with one of its aspects, provides vinorelbine liposome preparations which have been dehydrated in the presence of one or more protective sugars. In certain preferred embodiments of the invention, the liposomes are dehydrated with the one or more sugars being present at both the inside and outside surfaces of the liposome membranes. In other preferred embodiments, the sugars are selected from the group consisting of trehalose, maltose, lactose, sucrose, glucose, and dextran, with the most preferred sugars from a performance point of view being trehalose and sucrose. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration.

The dehydration is done under vacuum and can take place either with or without prior freezing of the liposome preparation. The liposomes are preferably dehydrated using standard freeze-drying equipment or equivalent apparatus, that is, they are preferably dehydrated under reduced pressure. If desired, the liposomes and their surrounding medium can be frozen in liquid nitrogen before being dehydrated. Alternatively, the liposomes can also be dehydrated without prior freezing, by simply being placed under reduced pressure.

It has been found that inventive liposomes having a concentration gradient across their membranes can be dehydrated in the presence of one or more sugars, stored in their dehydrated condition, subsequently rehydrated, and the concentration gradient then used to create a transmembrane potential which will load vinorelbine into the liposomes. Alternatively, the concentration gradient can be created after the liposomes have been dehydrated, stored, and rehydrated.

When the dehydrated liposomes are to be used, rehydration is accomplished by adding diluent, such as water for injection, normal saline, 5% dextrose in normal saline (D5W). The vinorelbine liposomes can be resuspended into the aqueous solution by gentle swirling of the solution. The rehydration can be performed at room temperature or at other temperatures appropriate to the composition of the liposomes and their internal contents.

The invention includes pharmaceutical preparations that in addition to non-toxic, inert pharmaceutically suitable excipients contain the liposome-entrapped vinorelbine and processes for the production of these preparations.

The invention also includes pharmaceutical preparations in dosage units. This means that the preparations are in the form of individual parts, for example capsules, softgel capsules, pills, suppositories, ampoules, and vials, of which the content of liposome entrapped vinorelbine corresponds to a fraction or a multiple of an individual dose. The dosage units can contain, for example, 1, 2, 3, or 4 individual doses or ½, ⅓ or ¼ of an individual dose. An individual dose preferably contains the amount of vinorelbine which is given in one administration and which usually corresponds to a whole, a half or a third or a quarter of a daily dose.

The abovementioned pharmaceutical preparations are manufactured in the usual manner according to known methods, for example by mixing liposomal vinorelbine with an excipient or excipients. By non-toxic, inert pharmaceutically suitable excipients there are to be understood solid, semi-solid or liquid diluents, fillers, solubilizers, stabilizer and formulation auxiliaries of all kinds.

The active compound or its pharmaceutical preparations administered locally, orally, parenterally, intraperitoneally and/or rectally, preferably parenterally, especially intravenously. Suitable amounts are therapeutically effective amounts that do not have excessive toxicity, as determined in empirical studies. Accordingly, any pharmaceutical preparation suitable to the desired route of administration, e.g., tablets, dragees, capsules, pills, granules, suppositories, solutions, suspensions and emulsions, pastes, ointments, gels, creams, lotions, powders, and sprays, can be used. Suppositories can contain, in addition to the liposome-entrapped vinorelbine, suitable water-soluble or water-insoluble excipients. Suitable excipients are those in which the inventive liposomal entrapped vinorelbine are sufficiently stable to allow for therapeutic use, for example polyethylene glycols, certain fats, and esters or mixtures of these substances. Ointments, pastes, creams, and gels can contain suitable excipients in which the liposome-entrapped vinorelbine is stable and can contain additives such as eucalyptus oil and sweeteners like saccharin.

The present invention also includes the use of the active compound according to the invention and of pharmaceutical preparations which contain the active compound according to the invention in human and veterinary medicine for the prevention, amelioration and/or cure of diseases, in particular those diseases caused by cellular proliferation, such as cancer, in any mammal, such as a cow, horse, pig, dog, or cat. For example, dog lymphoma can be treated effectively with the present vinorelbine formulation. However, the present formulation is particularly preferred for use in the treatment of human patients, particularly for cancer and other diseases caused by cellular proliferation. The inventive compositions have particular use in treating human lymphoma, ovarian, breast, lung (e.g., unresectable, advanced non small cell lung cancer), and colon cancers.

The vinorelbine should preferably be present in the abovementioned pharmaceutical preparations in a concentration of about 0.1 to 50, preferably of about 0.5 to 25, percent by weight of the total mixture. Depending, in part, on the route of administration, the usual initial dose of vinorelbine is about 25-60 mg/m². In a human, for example, preferably, about 25-40 mg/m² is administered. However, it can be necessary to deviate from the dosages mentioned and in particular to do so as a function of the nature and body weight of the subject to be treated, the nature and the severity of the illness, the nature of the preparation and the administration of the medicine, and the time or interval over which the administration takes place. Thus it can suffice in some cases to manage with less than the abovementioned amount of active compound while in other cases the abovementioned amount of active compound can be exceeded. However, determining an optimal dosage is within the ordinary skill of a practitioner in this field, and the particular required optimum dosage and the type of administration of the vinorelbine can be determined by one skilled in the art, by available methods.

One significant advantage of the present composition is that it provides a method of modulating multidrug resistance in cancer cells that are subjected to vinorelbine. In particular, the present liposomal compositions reduce the tendency of cancer cells subjected to chemotherapy with vinorelbine to develop resistance thereto, and reduce the tendency of treated cells of developing resistance to other therapeutic agents, such as cisplatin, vindesine, taxol, 5-fluorouracil (5-FU), or leucovorin, for example. Thus, other agents can be advantageously employed with the present treatment either in the form of a combination active with vinorelbine or by separate administration. Another advantage of the present composition is that the present liposomal compositions reduce the irritation, local tissue necrosis, and/or thrombophlebitis. By using the present liposomal compositions, the extravasation injuries is significantly reduced since the free vinorelbine is not in contact with the tissue directly. Moreover, in some applications of the inventive method, approximately 3-fold less vinorelbine accumulates in cardiac tissue, as compared to the administration of the same amount of vinorelbine in a conventional vinorelbine formulation, when measured by conventional methods. Moreover, the area under the vinorelbine plasma concentration curve is 200-fold higher in some applications of the inventive method as compared to the area when a conventional vinorelbine formulation is administered in a conventional manner. Furthermore, the inventive method can result in the vinorelbine plasma half-life being approximately 10-fold greater than with the conventional vinorelbine formulation.

Having described the present invention, reference will now be made to certain examples which are provided solely for purposes of illustration and which are not intended to be limiting.

### EXAMPLE 1

Vinorelbine (3 μmoles) is dissolved in chloroform containing 3 μmoles cardiolipin. To this mixture, 14 μmoles of phosphatidyl choline dissolved in hexane and 10 μmoles cholesterol in chloroform is added. The mixture is stirred gently and the solvents are evaporated under vacuum at below 30° C. to form a thin dry film of lipid and drug. Liposomes then are formed by adding 2.5 ml of saline solution and aggressively mixing the components by vortexing. The flasks then are vortexed to provide multilamellar liposomes and optionally sonicated in a sonicator to provide small unilamellar liposomes.

The efficiency of vinorelbine encapsulation is then determined by dialysis of an aliquot of the liposomal preparation overnight in an aqueous solution and thereafter dissolving the liposomes in methanol and analyzing the sample by standard methods using high pressure liquid chromatography (HPLC), such as reverse phase HPLC or spectrophotometric method using UV/Vis spectrophotometer. Alternatively liposomes can be centrifuged at 50,000×g for 1 hour prior to dissolving them in methanol. Generally the encapsulation efficiency of vinorelbine in liposomes is more than 80% of the initial input dose.

### EXAMPLE 2

Similar experimental conditions as set forth in Example 1 can be employed with varying quantities of drug and lipid. For example, concentrations of 6 μM vinorelbine, 6 μM cardiolipin, 28 μM phosphatidyl choline, and 20 μM cholesterol can be used by dissolving them in a suitable solvent, evaporating the solvent, and dispersing the dried lipid/drug film in a suitable aqueous solvent such as 5 ml of 7% trehalose-saline solution. Hydration of the liposomes can be facilitated by vortexing and/or sonicating the mixture. The liposomes can then be dialyzed, as desired, and the percent encapsulation of vinorelbine in liposomes measured as described above. Typically, vinorelbine encapsulation should be 80% or more as assayed by HPLC or UV/Vis method.

### EXAMPLE 3

Vinorelbine can be entrapped in liposomes by using 3 μM of the drug, 15 μM of dipalmitoyl phosphatidyl choline, 1 μM cardiolipin, and 9 μM cholesterol in a volume of 2.5 ml. The drug and lipid mixture can be evaporated under vacuum and resuspended in an equal volume of saline solution. The remainder of the process is similar to that described above. The vinorelbine encapsulation efficiency will generally be higher than 80% in this system.

### EXAMPLE 4

In this preparation of liposomes, 2 μM vinorelbine, 2 μM of phosphatidyl serine, 11 μM phosphatidylcholine, 2 μM cardiolipin, and 7 μM cholesterol are used. The entire process is as described above. Greater than 80% vinorelbine encapsulation efficiency can be expected.

### EXAMPLE 5

Vinorelbine (3 μmoles) is dissolved in chloroform containing 3 μmoles cardiolipin and the mixture allowed to form complexes. To facilitate complex formation the chloroform solvent is removed by evaporation. To the dry film 14 μmoles of phosphatidylcholine dissolved in hexane and 10 μmoles cholesterol in chloroform is added. The mixture is stirred gently, and the solvents evaporated under vacuum at below 30° C. to form a thin dry film of lipid and drug. Liposomes then are formed by adding 2.5 ml of saline solution and aggressively mixing the components by vortexing. The flasks then are vortexed to provide multilamellar liposomes and can optionally be sonicated in a sonicator to provide small unilamellar liposomes.

The efficiency of vinorelbine encapsulation is determined by dialysis of an aliquot of the liposomal preparation overnight in an aqueous solution and thereafter dissolving the liposomes in methanol and analyzing the sample by standard methods using high pressure liquid chromatography (HIPLC), such as reverse phase IIPLC. Alternatively liposomes can be centrifuged at 50,000×g for 1 hour prior to dissolving them in methanol. Generally the encapsulation efficiency of vinorelbine in liposomes will be more than 80% of the initial input dose.

### EXAMPLE 6

Vinorelbine liposome can be prepared using the following procedure: the lipids are mixed with the cardiolipin. The mixed powdered lipids are dissolved in chloroform in a round bottomed flask. The clear solution can be placed on a Buchi rotary evaporator at 30° C. for 30 min to make a thin film. The flask containing the thin lipid film then is dried under vacuum for 30 min. The film is then hydrated in vinorelbine aqueous solution containing sucrose. The hydrated lipid film is rotated in a 50° C. The mixture in the flask is votexed and mixed. The mixture is extruded sequentially through decreasing size filters: 800 nm, 400 nm, 200 nm, and 100 nm. The vinorelbine liposomes then are lyophilized under vacuum. The resulting dehydrated liposomes can be stored at 2-8° C. for at least 12 months. Prior to administration, the vinorelbine liposomes can be rehydrated by adding suitable diluent.

All of the references cited herein, including patents, patent applications, and publications, are hereby incorporated in their entireties by reference.

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the claims.

What is claimed is:

1. A method of treating a cellular proliferative disease, comprising administering to a mammalian host a pharmaceutical composition comprising:
(a) a therapeutically effective amount of liposomal vinorelbine also comprising cardiolipin, and
(b) a pharmaceutically acceptable excipient.

2. The method of claim 1, wherein the liposomal vinorelbine has an encapsulation efficicncy of at least about 80%.

3. The method of claim 1, wherein the liposomal vinorelbine further includes α-tocopherol.

4. The method of claim 1, wherein said mammalian host is a human.

5. The method of claim 1, wherein said cardiolipin is selected from the group consisting of natural cardiolipin and synthetic cardiolipin.

6. The method of claim 1, wherein said liposome bears a negative charge.

7. The method of claim 1, wherein said liposome bears a positive charge.

8. The method of claim 1, wherein said liposome is neutral.

9. The method of claim 1, wherein at least a portion of said vinorelbine is complexed with cardiolipin.

10. The method of claim 1, wherein said liposomes are a mixture of multilamellar vesicles and unilamellar vesicles.

11. The method of claim 1, wherein said pharmaceutical composition further comprises one or more therapeutic agents other than vinorelbine.

12. The method of claim 11, wherein one or more of said agents is an antineoplastic, antifungal, or antibiotic agent.

13. A therapeutic composition comprising liposomal vinorelbine comprising a first liposome forming material comprising cardiolipin and a second liposome forming material.

14. The composition of claim 13, wherein the liposomal vinorelbine has an encapsulation efficiency of at least about 80%.

15. The composition of claim 13, which further includes α-tocopherol.

16. The composition of claim 13, wherein a portion of said cardiolipin is complexed with said vinorelbine.

17. The composition of claim 13, wherein said liposome entrapped vinorelbine comprises vesicles having a diameter of about 5 μm or less.

18. The composition of claim 13, wherein said liposome entrapped vinorelbine comprises vesicles having a diameter of about 1 μm or less.

19. The composition of claim 13, wherein said liposome entrapped vinorelbine comprises vesicles having a diameter of about 0.5 μm or less.

20. The composition of claim 13, wherein said liposome entrapped vinorelbine comprises vesicles having a diameter of about 0.1 μm or less.

21. The composition of claim 13, wherein said second liposome-forming material is a lipid selected from the group consisting of phosphatidylcholine, cholesterol, α-tocopherol, dipalmitoyl phosphatidylcholine and phosphatidyl serine.

22. The composition of any of claims 13, wherein said cardiolipin is selected from the group consisting of natural cardiolipin and synthetic cardiolipin.

23. The composition of claim 13, wherein said liposome bears a negative charge.

24. The composition of claim 13, wherein said liposome bears a positive charge.

25. The composition of claim 13, wherein said liposome is neutral.

26. The composition of claim 13, wherein said liposome is a mixture of multilamellar vesicles and unilamellar vesicles.

27. The composition of claims 13; wherein said pharmaceutical composition further comprises one or more therapeutic agents other than vinorelbine.

28. The composition of claim 27, wherein one or more of said agents is an antineoplastic, antifungal, or antibiotic agent.

29. The composition of claim 13, further comprising one or more pharmaceutically acceptable excipients.

30. The composition of claim 29, wherein one or more of said excipients enhances shelf-life of the composition.

31. The composition of claim 29, wherein one or more of said excipients improves the stability of the composition.

32. The composition of claim 29, wherein one or more of said excipients is a sugar.

33. The composition of claim 32, wherein the sugar is selected from the group consisting of trehalose, maltose, sucrose, glucose, lactose, and dextran.

34. The composition of claim 32 wherein the sugar is trehalose.

35. The composition of claim 32 wherein the sugar is sucrose.

36. The composition of claim 32 wherein the sugar is an aminoglycoside.

37. The composition of claim 36 wherein the aminoglycoside is streptomycin.

38. The composition of claim 36 wherein the aminoglycoside is dihydrostreptomycin.

39. The composition of claims 13 in dehydrated form.

40. The composition of claim 39, which is lyophilized.

41. The composition of claim 13, which is stable for up to about 12 months at between about 2° C. and about 8° C.

42. A method for the treatment of mammalian cancer comprising administering a therapeutically effective amount of the composition of claim 13 to a patient in need thereof.

43. A method for the treatment of mammalian cancer comprising administering a therapeutically effective amount of the composition of claim 27 to a patient in need thereof.

44. The method of claim 42, wherein the patient is human.

45. The method of claim 43, wherein the patient is human.
